(19) United States
(12) Patent Application Publication
(10) Pub. No.: US 2018/0154088 A1
(43) Pub. Date: Jun. 7, 2018

(54) METHOD OF VERIFYING AND LOGGING SYRINGE DRUG DOSAGES IN REAL TIME

(71) Applicant: eBroselow, LLC, Blacksburg, VA (US)

(72) Inventor: James B. Broselow, Hickory, NC (US)

(21) Appl. No.: 15/366,150

(22) Filed: Dec. 1, 2016

(51) Int. Cl. A61M 5/315; A61M 5/31

(52) U.S. Cl. CPC A61M 5/31566; A61M 5/3129; A61M 5/31573; A61J 1/2096; A61M 2205/6009; A61M 2205/50; A61M 2205/6072

(57) ABSTRACT

A method of verifying the correct amount of a drug in a syringe, including the steps of providing a handheld electronic barcode scanning device containing digital data that includes a range of medically appropriate drug dosages to be administered to a patient by a syringe and a range of positions of a syringe plunger in relation to a syringe barrel correlated to the medically appropriate drug dosages. A drug container is scanned that causes the handheld device to display the identity of the drug and medically appropriate drug dosages. A medically appropriate drug dosage for a specific patient is determined and drawn into a syringe. The syringe is scanned and digitally compares the position of the plunger in the syringe barrel with the range of positions of the syringe plunger stored as digital data in the handheld device to verify the correct amount of drug in the syringe based on the position of the plunger.

### TECHNICAL FIELD AND BACKGROUND OF THE INVENTION

This invention relates to medical treatments that include administering drugs in liquid form to a patient by means of a syringe. The invention is particularly useful in acute drug administration situations where encyclopedic medical references are unavailable and where memory and manual math calculations may be unreliable. The variety and complexity of modern medications increases the opportunity for errors to occur, threatening patient safety and imposition of liability on caregivers.

A syringe typically includes a barrel in which is positioned a plunger that is partially withdrawn from the barrel to draw a liquid into the barrel. The plunger is, then pushed back into the barrel to dispense the liquid contents from the barrel into the patient, most typically by means of a hollow needle or other dispensing orifice. The correct dosage is determined by drawing liquid into the barrel while visually noting the position of the liquid in the barrel in relation to a scale of graduated markings on the barrel corresponding to the desired dosage.

Medications are labeled with U.S. National Drug Codes (“NDC codes”). These codes provide a pathway into a database containing critical dosing and administration information for any patient that can be used to carry out the functions of this invention.

Substantial improvements in accurately determining correct drug dosages have been made in recent years as disclosed in, for example, U.S. Pat. Nos. 4,713,888; 4,823,469; 8,494,875; 8,676,602 and 6,764,469 of the present applicant. Color-coding ranges of drug dosages and equipment sizes have improved the ability of medical personnel to treat patients with medically appropriate procedures and to administer medically appropriate dosages of drugs. Correlating body weight with drug dosages and equipment sizes enables medical personnel to use information stored in databases to improve patient treatments, particularly in emergency situations.

However, even though a correct dosage is determined, it is still possible to draw the wrong amount of drug into a syringe, in which case an incorrect dosage is administered to the patient notwithstanding that information regarding a correct dosage was conveyed to the person administering the drug. Even though the incorrect dosage is administered, the patient chart may nevertheless be noted as the correct dosage having been administered.

### SUMMARY OF THE INVENTION

Therefore, it is an object of the invention to provide a means of providing essential information needed to safely administer acute medications at the point of care and to verify that a correct amount of a drug has been drawn into a syringe prior to administration to a patient.

It is another object of the invention to provide a method to quickly access critical dosing and administration information stored in a digital device without access to the internet.

It is another object of the invention to provide a simple way to update a patient chart in real time with the correct dosage of a drug administered by a syringe.

These and other objects and advantages of the present invention are achieved by a method of verifying the correct amount of a drug in a syringe that includes the steps of providing a handheld electronic barcode scanning device containing digital data that includes a range of medically appropriate drug dosages to be administered to a patient by a syringe and a range of positions of a syringe plunger in relation to a syringe barrel correlated to the medically appropriate drug dosages. A drug container is scanned, wherein the container includes an image code, that when scanned causes the handheld device to display the identity of the drug and medically appropriate drug dosages. The medically appropriate drug dosage for a specific patient is determined, and the medically appropriate drug dosage for a specific patient is drawn into a syringe. The syringe is scanned and the position of the plunger in the syringe barrel is compared with the range of positions of the syringe plunger stored as digital data in the handheld device to verify the correct amount of drug in the syringe based on the position of the plunger. The handheld device generates an output indicative of whether the amount of drug in the syringe is the correct amount based on the position of the syringe plunger.

According to another aspect of the invention, the drug container image code is based on U.S. National Drug Code.

According to another aspect of the invention, the step of scanning the drug container includes scanning with a laser.

According to another aspect of the invention, the step of scanning the syringe includes the step of photographing the syringe and storing the photograph as digital data in the handheld device.

According to another aspect of the invention, the method includes the step of adding a time stamp to digital data representing the scanned image of the syringe.

According to another aspect of the invention, the drug container image code is a QR, UPC-A, EAN-8, EAN-13, code39, code128 or ITF barcode.

According to another aspect of the invention, the range of positions of the syringe plunger stored as digital data in the handheld device includes a variation tolerance of ±10 percent of the correct amount of drug in the syringe.

According to another aspect of the invention, the method includes the steps of coding the barrel of the syringe with an array of colors along the barrel and correlating the digital data representing the range of positions of the syringe plunger with the array of colors, wherein the step of comparing the position of the plunger in the syringe barrel with the range of positions of the syringe plunger stored as digital data in the handheld device includes the step of displaying on the handheld device a single color of the array of colors corresponding to the medically appropriate drug dosage.

According to another aspect of the invention, a method of verifying the correct amount of a drug in a syringe includes the steps of providing a handheld electronic barcode scanning device containing digital data that includes a range of medically appropriate drug dosages to be administered to a patient by a syringe and a range of positions of a syringe plunger in relation to a syringe barrel correlated to the medically appropriate drug dosages; scanning with a laser a drug container having a U.S. National Drug Code that when scanned, causes the handheld device to display the identity of the drug and medically appropriate drug dosages; determining the medically appropriate drug dosage for a specific patient; drawing the drug into a syringe; photographing the syringe and storing the photograph as digital data in the handheld device; adding a time stamp to digital data representing the scanned image of the syringe; digitally comparing the position of the plunger in the syringe barrel with the range of positions of the syringe plunger stored as digital data in the handheld device to verify the correct amount of drug in the syringe based on the position of the plunger indicated by the scanned image of the syringe, wherein the range of positions of the syringe plunger stored as digital data in the handheld device includes a variation tolerance of ±10 percent of the correct amount of drug in the syringe, and the handheld device generating an output indicative of whether the amount of drug in the syringe is the correct amount based on the position of the syringe plunger.

According to another aspect of the invention, the method includes the steps of coding the barrel of the syringe with an array of colors along the barrel and correlating the digital data representing the range of positions of the syringe plunger with the array of colors, wherein the step of comparing the position of the plunger in the syringe barrel with the range of positions of the syringe plunger stored as digital data in the handheld device includes the step of displaying on the handheld device a single color of the array of colors corresponding to the medically appropriate drug dosage.

According to another aspect of the invention, the method includes the step of scanning a patient-specific image code that includes the patient's weight.

According to another aspect of the invention, the patient-specific image code is contained on an object selected from the group consisting of a patient armband, chart and medication label.

### BRIEF DESCRIPTION OF THE DRAWINGS

FIG. 1 is a view of a handheld digital data storage device and scanner in the process of scanning a barcode-labeled drug vial;

FIG. 2 is a view of a drug being dispensed from the vial into a syringe;

FIG. 3 is a view of the syringe being scanned to determine the position of the syringe plunger or to determine the capacity of the syringe;

FIG. 4 is a view of the handheld digital storage device and scanner transmitting dosage information to a patent chart handheld device from, for example a syringe;

FIG. 5 is a view of the handheld digital storage device and scanner syringe dosage and the patent chart handheld device showing that both handheld devices display the same information;

FIG. 6 is a view showing an alternative embodiment of the invention that utilizes colored zones on a syringe barrel; and

FIG. 7 is a view showing that by scanning the syringe an indication is displayed on the handheld device that the correct drug and dosage has been drawn into the syringe.

### DETAILED DESCRIPTION OF PREFERRED EMBODIMENT

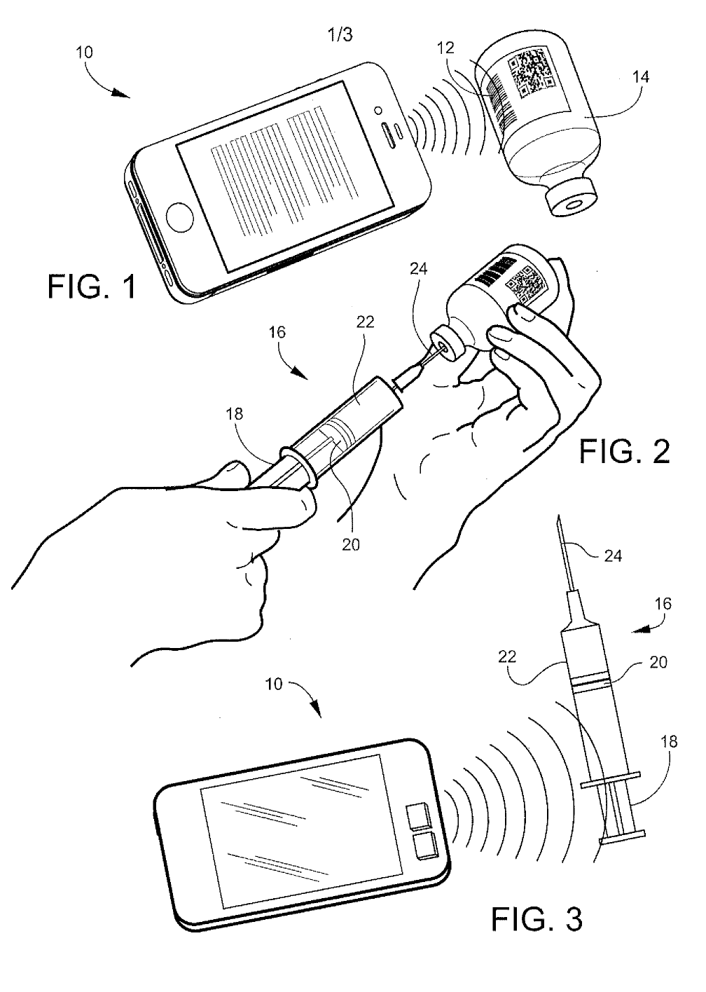

Referring now to the drawings, FIG. 1 shows a handheld device 10 that includes both digital storage capability and a laser scanner capable of scanning codes such as QR, UPC-A, EAN-8, EAN-13, code39, code128 and ITF barcodes, among other suitable barcodes. (“image codes”). Devices suitable for use include Honeywell CT50, Android 4.4.4 KITKAT Healthcare version; Honeywell 75E, Android KITKAT and iPad Generations 5 and 6 and iPhone 5 and 5E Apple devices with available healthcare sleds.

The image code 12 of FIG. 1 is applied as a label to a drug container, such as a vial. Scanning the image code 12 with scanning software stored in the handheld device 10 accesses drug dosage data stored as digital data accessible by the handheld device 10 and displays the appropriate drug dosage information.

If, for example, the dosage for a specific patient is 3.5 mLs, that amount of liquid is withdrawn from the vial 14 into a syringe 16 by withdrawing the plunger 18 to a position where the rubber plunger head 20 is positioned proximate the correct position in the barrel 22, as shown in FIG. 2. In current practice, the drug would then be injected into the body of a patient through the needle 24 by depressing the plunger with the thumb, forcing the drug from the barrel 22 through the needle 24.

In accordance with the invention, before the drug is administered, the handheld device 10 is switched to camera mode and the syringe 16 is scanned and photographed. The handheld device 10 includes a database that contains data comprising a range of positions of the syringe plunger 18 in relation to the syringe barrel 22, which in turn is correlated to medically appropriate drug dosages. The database may contain a file of images which software compares with the scanned or photographed image of the plunger 18 in relation to the barrel 22. Thus, the position of the plunger 18 enables the handheld device 10 to determine whether the plunger 18 is in the expected position for the desired drug dosage, for example, 3.5 mLs, with a small allowance for variance such as ±10 percent. The photograph of the syringe is stored and becomes a part of the patient's medical record, documenting that the correct drug dosage was actually loaded into the syringe and administered to the patient.

Alternatively or in addition to the determination of the correct plunger 18 position, the database may also contain images of syringes of different capacity, so that scanning the syringe barrel 22 either confirms that the correct capacity syringe is being used or alerts the user that an error in syringe capacity and thus, the amount of drug drawn into the syringe has occurred.

In addition to the handheld device 10 being able to scan the vial 14 to input the drug information, it can also input the patient's weight by scanning a patient's identification armband, patient specific medication label, PC screen, Pyxis screen or other source that contains information regarding the patient's weight, such as by an image code 12. As disclosed in the above-referenced patents, weight-based dosing is an important improvement in drug safety.

The above procedures are preferably “off line” and maybe used at, for example, accident sites, in emergency vehicles and critical care facilities where ready access to drug dosage and equipment usage information can be readily stored on the handheld device 10 for use without access to the internet.

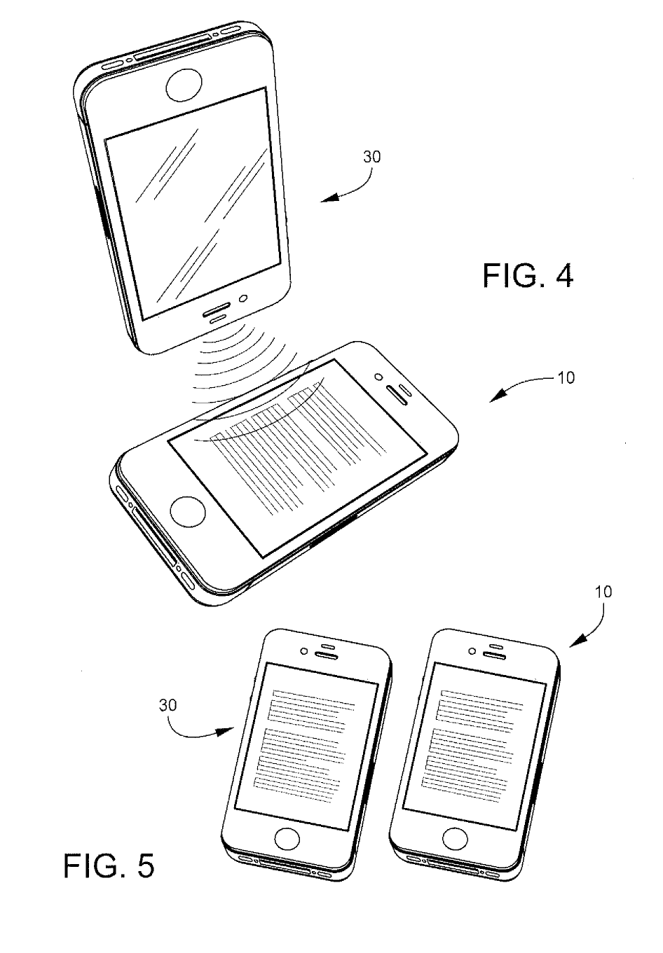

Referring now to FIGS. 4 and 5, upon arrival at a hospital, clinic or other care facility, the handheld device 10 can be used to transfer information regarding the correct syringe dosage to a computer, which may be another handheld device 30 or any other image-capturing device that becomes part of the patient record. This can be accomplished by converting the information to be transferred into an image code, such as a barcode or QR code that is displayed on the screen of the handheld device 10. By photographing the image code with the handheld device 30, the information captured by the handheld device 10 can be transmitted easily and accurately and logged with a time stamp indicating when the syringe was scanned.

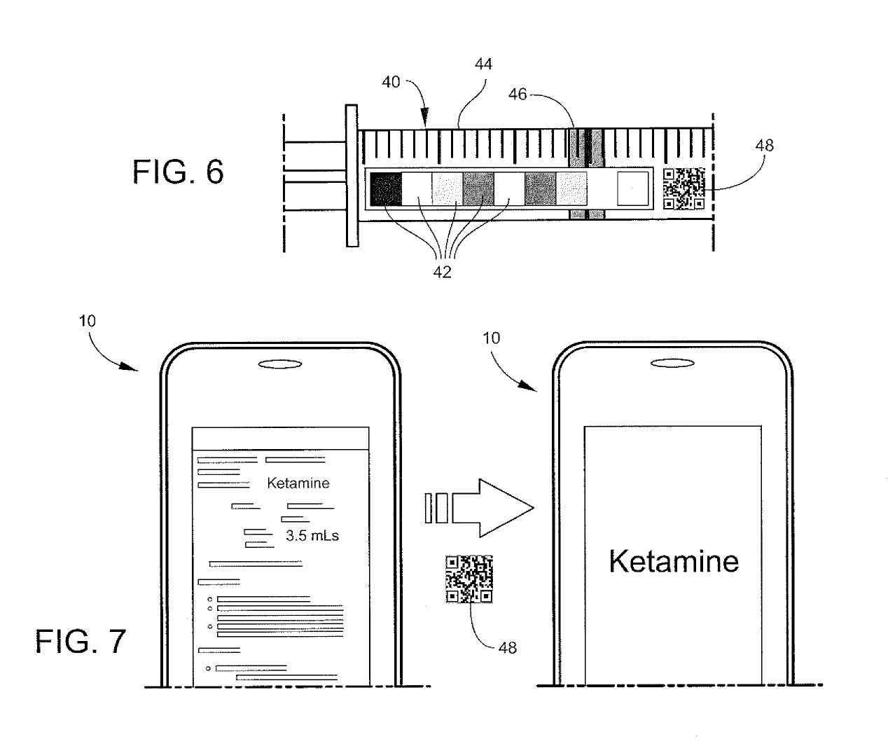

Referring now to FIG. 6, a syringe 40 is shown that includes an array of colors 42 extending along the length of the syringe barrel 44. Rather than scanning the syringe 40 to determine the position of the plunger, the barrel 44 is scanned and the position of the plunger head 46 is compared with information stored in the handheld device 10 that correlates the position of the plunger head 46 with one of the colors on the syringe barrel 44. An image code, such as the QR code 48 positioned at one end of the array of colors 42 determines the correlation of the plunger head 46 with the correct drug dosage for the specific drug being administered by the syringe 40. As shown in FIG. 7, scanning the syringe barrel 44 displays a screen on the handheld device 10 that indicates the drug being administered, for example, Ketamine, and the screen color corresponds to the position of the plunger head 46 on the syringe barrel 44, for example, in the purple zone.

According to a further iteration, a patient weight can be set by scanning the image code 12 or 48 containing a drug dosage for a patient having a weight falling within one of several weight ranges, which may be color-coded. The vial 14 is then scanned and a dosage is determined based on the patient weight. The indicated dosage, typically in mLs, is drawn into the syringe 16 or 40. The syringe 16 or 40 is then scanned as described above. The screen of the handheld device 10 displays a screen as shown in FIG. 7 that verifies the correct dosage by displaying the drug and dosage detected by the scanner. The user then compares the display of the handheld device 10 with the syringe 16 or 40 to determine whether the correct dosage has been drawn from the vial 12. The scanning can be carried out by a laser scanner or by using the still photo or video feature of the handheld device 10. As an additional accuracy check, another clinician or technician can observe the display and confirm that the correct dosage has been drawn into the syringe 16 or 40. When scanning the syringe 40, the handheld device 10 can be programmed to display a screen color, such as a background color, that matches the color on the syringe barrel 44 aligned with the plunger head 46.

In each of the variations described above any clinician or other user, such as a pharmacist or pharmacy technician, can use the procedures and techniques to determine correct syringe contents or capacity as a failsafe against incorrect drug administration. For example, as a validation step, weight is determined by scanning a patient armband, medication container label or display screen and sending the scanned image together with a photograph of the display screen of the handheld device 10 to the pharmacist for validation. An image of the diluent label can be sent as well. The pharmacist can simply reply with text indicating agreement.

In the above discussion and descriptions a syringe has been used as the primary example of the practice of the invention. However, other drug delivery devices, such as tubes and the like for oral administration are also within the scope of the invention, the purpose being to provide a safety check on the contents of a delivery device after the device has been loaded with a drug and just prior to administration.

A method of verifying and logging syringe drug dosages in real time according to the invention have been described with reference to specific embodiments and examples. Various details of the invention maybe changed without departing from the scope of the invention. Furthermore, the foregoing description of the preferred embodiments of the invention and best mode for practicing the invention are provided for the purpose of illustration only and not for the purpose of limitation, the invention being defined by the claims.

I claim:

1. A method of verifying the correct amount of a drug in a syringe, comprising the steps of:
(a) providing a handheld electronic barcode scanning device containing digital data that includes a range of medically appropriate drug dosages to be administered to a patient by a syringe and a range of positions of a syringe plunger in relation to a syringe barrel correlated to the medically appropriate drug dosages;
(b) scanning a drug container having an image code that when scanned causes the handheld device to display the identity of the drug and medically appropriate drug dosages;
(c) determining the medically appropriate drug dosage for a specific patient;
(d) drawing the medically appropriate drug dosage for a specific patient into a syringe;
(e) scanning the syringe and digitally comparing the position of the plunger in the syringe barrel with the range of positions of the syringe plunger stored as digital data in the handheld device to verify the correct amount of drug in the syringe based on the position of the plunger; and
(f) the handheld device generating an output indicative of whether the amount of drug in the syringe is the correct amount based on the position of the syringe plunger.

2. A method according to claim 1, wherein the drug container image code comprises drug identification based on U.S. National Drug Code.

3. A method according to claim 1, wherein the step of scanning the drug container comprises scanning with a laser.

4. A method according to claim 1, wherein the step of scanning the syringe comprises the step of photographing the syringe and storing the photograph as digital data in the handheld device.

5. A method according to claim 1, and including the step of adding a time stamp to digital data representing the scanned image of the syringe.

6. A method according to claim 1, wherein the drug container image code comprises a QR, UPC-A, EAN-8, EAN-13, code39, code128 or ITF barcode.

7. A method according to claim 1, wherein the range of positions of the syringe plunger stored as digital data in the handheld device includes a variation tolerance of ±10 percent of the correct amount of drug in the syringe.

8. A method according to claim 1, and including the steps of coding the barrel of the syringe with an array of colors along the barrel and correlating the digital data representing the range of positions of the syringe plunger with the array of colors, wherein the step of comparing the position of the plunger in the syringe barrel with the range of positions of the syringe plunger stored as digital data in the handheld device includes the step of displaying on the handheld device a single color of the array of colors corresponding to the medically appropriate drug dosage.

9. A method of verifying the correct amount of a drug in a syringe, comprising the steps of:
(a) providing a handheld electronic barcode scanning device containing digital data that includes a range of medically appropriate drug dosages to be administered to a patient by a syringe and a range of positions of a syringe plunger in relation to a syringe barrel correlated to the medically appropriate drug dosages;
(b) scanning with a laser a drug container having a U.S. National Drug Code that when scanned causes the handheld device to display the identity of the drug and medically appropriate drug dosages;
(c) determining the medically appropriate drug dosage for a specific patient;
(d) drawing the drug into a syringe;
(e) photographing the syringe and storing the photograph as digital data in the handheld device;
(f) adding a time stamp to digital data representing the scanned image of the syringe;
(g) digitally comparing the position of the plunger in the syringe barrel with the range of positions of the syringe plunger stored as digital data in the handheld device to verify the correct amount of drug in the syringe based on the position of the plunger indicated by the scanned image of the syringe, wherein the range of positions of the syringe plunger stored as digital data in the handheld device includes a variation tolerance of ±10 percent of the correct amount of drug in the syringe; and
(h) the handheld device generating an output indicative of whether the amount of drug in the syringe is the correct amount based on the position of the syringe plunger.

10. A method according to claim 9, and including the steps of coding the barrel of the syringe with an array of colors along the barrel and correlating the digital data representing the range of positions of the syringe plunger with the array of colors, wherein the step of comparing the position of the plunger in the syringe barrel with the range of positions of the syringe plunger stored as digital data in the handheld device includes the step of displaying on the handheld device a single color of the array of colors corresponding to the medically appropriate drug dosage.

11. A method according to claim 9, and including the step of scanning a patient-specific image code that includes the patient's weight.

12. A method according to claim 11, wherein the patient-specific image code is contained on an object selected from the group consisting of a patient armband, chart and medication label.

13. A method according to claim 11, wherein the step of generating an output indicative of whether the amount of drug in the syringe is the correct amount based on the position of the syringe plunger includes the step of transmitting the information including the time stamp to a patient chart.

14. A method according to claim 11, including the step of validating the correct dosage administered to a patient by determining the weight of the patient by scanning a patient armband, medication container label or display screen, transmitting the scanned image together with a photograph of the display screen of the handheld device to a pharmacist for validation.
